SEQUENCE LISTING (20 sequences)

SEQ ID NO: 1 - protein, 112 aa, HOMO SAPIENS
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 2 - protein, 394 aa, HOMO SAPIENS
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 3 - protein, 11 aa, ARTIFICIAL
  His-6 purification tag
HHHHHHGSGSG

SEQ ID NO: 4 - protein, 23 aa, ARTIFICIAL
  Linker sequence
GGSSSGSGGSGGSGSGGSGGSGS

SEQ ID NO: 5 - protein, 32 aa, ARTIFICIAL
  Linker sequence
GGSSSGSGGSGGSGSGGSGGSGSGSGGSGGSG

SEQ ID NO: 6 - protein, 32 aa, ARTIFICIAL
  Linker sequence
PPPPSGSGGSGGSGSGGSGGSGSGSGGSGGSG

SEQ ID NO: 7 - protein, 33 aa, ARTIFICIAL
  Linker sequence
PTPTPTPTPTPTPTPTPTPTPTPTPTPTPTPTP

SEQ ID NO: 8 - protein, 32 aa, ARTIFICIAL
  Linker sequence
EAAEAAEAAEAAEAAEAAEAAEAAEAAEAAEE

SEQ ID NO: 9 - protein, 36 aa, ARTIFICIAL
  Linker sequence
GGSSEAAEAAEAAEAAEAAEAAEAAEAAEAAEAAEE

SEQ ID NO: 10 - protein, 36 aa, ARTIFICIAL
  Linker sequence
PPPPEAAEAAEAAEAAEAAEAAEAAEAAEAAEAAEE

SEQ ID NO: 11 - protein, 23 aa, Artificial Sequence
  Linker sequencw
GGSSEAAEAAEAAEAAEAAEAAE

SEQ ID NO: 12 - protein, 23 aa, Artificial Sequence
  Linker sequence
GGSSPTPTPTPTPTPTPTPTPTP

SEQ ID NO: 13 - protein, 35 aa, Artificial Sequence
  Linker sequence
PPPEAAEAAEAAEAAEAAEAAEAAEAAEAAEAAEE

SEQ ID NO: 14 - protein, 34 aa, Artificial Sequence
  Linker sequence
PPEAAEAAEAAEAAEAAEAAEAAEAAEAAEAAEE

SEQ ID NO: 15 - protein, 39 aa, Artificial Sequence
  Linker sequence
EAAEAAEAAEAAEAAEAAEPEAAEAAEAAEAAEAAEAAE

SEQ ID NO: 16 - protein, 40 aa, Artificial Sequence
  Linker sequence
EAAEAAEAAEAAEAAEAAEPPEAAEAAEAAEAAEAAEAAE

SEQ ID NO: 17 - protein, 30 aa, Artificial Sequence
  Linker sequence
EAAAEAAAEAAAEAAAEAAAEAAAEAAAEE

SEQ ID NO: 18 - protein, 32 aa, Artificial Sequence
  Linker sequence
QAAQAAQAAQAAQAAQAAQAAQAAQAAQAAQQ

SEQ ID NO: 19 - protein, 112 aa, Homo sapiens
AREGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 20 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHAKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

(19) United States
(12) Patent Application Publication
(10) Pub. No.: US 2017/0334971 A1
(43) Pub. Date: Nov. 23, 2017

(54) Alpha-1-Antitrypsin (A1AT) Fusion Proteins and Uses Thereof

(71) Applicant: Novo Nordisk A/S, Bagsvaerd (DK)

(72) Inventor: Allan Christian Shaw, Copenhagen N (DK)

(73) Assignee: Novo Nordisk A/S, Bagsvaerd (DK)

(21) Appl. No.: 15/535,583

(22) PCT Filed: Dec. 22, 2015
(86) PCT No.: PCT/EP2015/080998

§ 371 Date: Jun. 13, 2017

(30) Foreign Application Priority Data
Dec. 22, 2014 (EP) 14199683.5

(51) Int. Cl. C07K 14/81; C07K 14/475; A61K 38/00

(52) U.S. Cl. CPC C07K 14/8125; C07K 14/475; A61K 38/00; C07K 2319/31

(57) ABSTRACT

The invention relates to MIC-1 fusion proteins. More specifically it relates to compounds comprising fusion proteins comprising a MIC-1 protein or an analogue thereof at the C-terminus of the fusion protein and a functional variant of human A1AT (A1AT) at the N-terminus of the fusion protein connected via a peptide linker. The compounds of the invention have MIC-1 activity. The invention also relates to pharmaceutical compositions comprising such compounds and pharmaceutically acceptable excipients, as well as the medical use of the compounds.

### TECHNICAL FIELD

The present invention relates to alpha-1-antitrypsin (A1AT) fusion proteins and their pharmaceutical use.

### INCORPORATION-BY-REFERENCE OF THE SEQUENCE LISTING

The Sequence Listing, entitled “SEQUENCE LISTING”, was created on 21 Dec. 2015 and is incorporated herein by reference.

### BACKGROUND

The macrophage inhibitory cytokine-1 (MIC-1), also known as GDF-15 and placental bone morphogenetic protein (PLAB), is a distant member of the TGF-beta super family, a family of peptide hormones involved in cell growth and differentiation. MIC-1 circulates as a cysteine-rich homodimer with a molecular mass of 24.5 kDa. MIC-1 was initially reported to be up-regulated in macrophages by stimuli including IL-1b, TNF-alpha, IL-2, and TGF-b. It was also shown that MIC-1 could reduce lipopolysaccharide-induced TNF-alpha production and it was based on these data proposed that MIC-1 was an anti-inflammatory cytokine. More recently, a study was investigating why human patients with advanced cancer were losing body weight and they showed that the weight loss correlated with circulating levels of MIC-1. These data indicates that MIC-1 regulates body weight. This hypothesis was tested in mice xenografted with prostate tumor cells, where data showed that elevated MIC-1 levels were associated with loss of body weight and decreased food intake, this effect being reversed by administration of antibodies to MIC-1. As administration of recombinant MIC-1 to mice regulated hypothalamic neuropeptide Y and pro-opiomelanocortin it was proposed that MIC-1 regulates food intake by a central mechanism. Furthermore, transgenic mice overexpressing MIC-1 are gaining less weight and body fat both on a normal low fat diet and on a high fat diet. Also, transgenic mice overexpressing MIC-1 fed both on a low and high fat diet, respectively, had improved glucose tolerance compared with wild type animals on a comparable diet.

Native MIC-1 has a short half-life, meaning that treatment with native MIC-1 requires daily administration to maintain efficacy.

US 2012/0094356 concerns the use of human alpha-1-antitrypsin or variants thereof for fusions to proteins and peptides of pharmaceutical interest.

WO 2005099746 concerns a method of modulating appetite and/or body weight by administering a MIC-1 modulating agent.

### SUMMARY

The present invention relates to development of biologically active recombinant fusion proteins comprising an N-terminal fusion partner fused to MIC-1 by means of intervening peptide linkers. The disclosed fusion proteins integrates a range of beneficial features such as improved production feasibility, protein stability, plasma half-life and a maintained biological activity of the fusion protein.

In one aspect, the invention provides compounds comprising fusion proteins comprising a MIC-1 protein or an analogue thereof at the C-terminus of the fusion protein and a functional variant of human alpha-1-antitrypsin (A1AT) at the N-terminus of the fusion protein connected via a peptide linker. The peptide linker has a length of 10 to 100 amino acids. In one aspect, the peptide linker comprises the amino acid sequence [X-Yₘ]ₙ, wherein X is Asp or Glu; Y is Ala; m is from 2 to 4, and n is at least 5.

In one aspect, the invention provides a polynucleotide molecule encoding a compound comprising a fusion protein comprising a MIC-1 protein or an analogue thereof at the C-terminus of the fusion protein and a functional variant of human A1AT at the N-terminus of the fusion protein connected via a peptide linker.

In one aspect, the invention provides a pharmaceutical composition comprising a compound of the invention or a pharmaceutically acceptable salt, amide or ester thereof, and one or more pharmaceutically acceptable excipients.

In one aspect, the invention provides a compound of the invention for use as a medicament.

In one aspect, the invention provides a compound of the invention for use in the treatment of eating disorders, such as obesity, e.g. by decreasing food intake, reducing body weight, suppressing appetite and inducing satiety.

In one aspect, the invention provides a compound of the invention for use in the treatment of obesity.

In one aspect, the compounds of the invention are MIC-1 agonists. In one aspect, the compounds of the invention inhibit food intake. In one aspect, the compounds of the invention reduce body weight.

In one aspect, the compounds of the invention have longer half-life than the half-life of native MIC-1.

### BRIEF DESCRIPTION OF DRAWINGS

FIG. 1. Schematic representation of an A1AT-MIC-1 dimeric fusion protein. A, B and C depicts relative positions of the A1AT domain, the linker region and MIC-1, respectively. -SS- indicates interchain disulphide bridge linking together the two A1AT-MIC-1 monomers to form a functional dimeric fusion protein.

### DESCRIPTION

The invention relates to compounds comprising MIC-1 fusion proteins. In one aspect, the invention relates to MIC-1 fusion proteins.

In one aspect, the invention provides compounds comprising fusion proteins comprising MIC-1 or an analogue thereof at the C-terminus of the fusion protein and human A1AT or a functional variant thereof at the N-terminus of the fusion protein connected via a peptide linker. The peptide linker has a length of 10 to 100 amino acids. In one aspect, the peptide linker comprises the amino acid sequence [X-Yₘ]ₙ, wherein X is Asp or Glu; Y is Ala; m is from 2 to 4, and n is at least 5.

The fusion protein strategy of the present invention combines the soluble, stable plasma protein human alpha-1-antitrypsin (A1AT) with native MIC-1. Human A1AT has inherent properties such as high solubility and stability which makes it beneficial to use as fusion partner for improving expression yield and conferring stability to MIC-1. Human A1AT as fusion partner may also increase the plasma half-life of MIC-1 by a significant size increase. The present invention provides compounds comprising MIC-1 fusion proteins with increased plasma half-life.

In what follows, Greek letters may be represented by their symbol or the corresponding written name, for example: α=alpha; β=beta; ε=epsilon; γ=gamma; ω=omega; etc. Also, the Greek letter of μ may be represented by “u”, e.g. in μl=ul, or in μM=uM.

### MIC-1 Proteins and Analogues

The term “MIC-1” as used herein means macrophage inhibitory cytokine-1 (MIC-1), also known as GDF-15, and placental bone morphogenetic protein (PLAB). The sequence of the full length wild type human MIC-1 protein is available from the UNIPROT database with accession no. Q99988. The 308 amino acid precursor protein includes a signal peptide (amino acids 1-29), a propeptide (amino acids 30-196) and a mature protein (amino acids 197-308). The 112 amino acid mature MIC-1 protein is included herein as SEQ ID NO:1. Mature MIC-1 contains nine cysteine residues which gives rise to the formation of 4 intrachain disulphide bonds and one interchain disulphide bond to create a covalently linked 24.5 kDa homodimer. A naturally occurring mutation corresponding to His6Asp in the mature protein (SEQ ID NO:1) has been described.

Thus particular examples of wild type human MIC-1 are the mature MIC-1 protein of SEQ ID NO:1, SEQ ID NO:1 having the amino acid modification His6Asp, as well as any of these sequences preceded by the propeptide and/or signal peptide referred to above.

The term “MIC-1 protein” as used herein refers to the human MIC-1 protein of SEQ ID NO:1, or an analogue thereof. The protein having the sequence of SEQ ID NO:1 may also be designated “native” MIC-1 or “wild type” MIC-1.

The term “MIC-1 analogue”, or “analogue of MIC-1 protein” as used herein refers to a protein, or a compound, which is a variant of the mature MIC-1 protein (SEQ ID NO:1). In one aspect, the MIC-1 analogue is a functional variant of the mature MIC-1 protein (SEQ ID NO:1). In one aspect of the invention, the MIC-1 analogues display at least 85%, 90% or 95% sequence identity to native MIC-1 (SEQ ID NO:1).

In another aspect of the invention, the MIC-1 analogues comprise less than 17 amino acid modifications (substitutions, deletions, additions (including insertions) and any combination thereof) relative to human native MIC-1 (SEQ ID NO:1). As an example of a method for determination of the sequence identity between two analogues the two peptides His6Asp MIC-1 and native MIC-1 are aligned. The sequence identity of the His6Asp MIC-1 analogue relative to native MIC-1 is given by the number of aligned identical residues minus the number of different residues divided by the total number of residues in native MIC-1. Accordingly, in said example the sequence identity is (112-1)/112.

The term “amino acid modification” used throughout this application is used in the meaning of a modification to an amino acid as compared to native MIC-1 (SEQ ID NO:1). This modification can be the result of a deletion of an amino acid, addition of an amino acid, substitution of one amino acid with another or a substituent covalently attached to an amino acid of the peptide.

Substitutions.

In one aspect amino acids may be substituted by conservative substitution. The term “conservative substitution” as used herein denotes that one or more amino acids are replaced by another residue having similar biophysical properties. Examples include substitution of amino acid residues with similar characteristics, e.g. small amino acids, acidic amino acids, polar amino acids, basic amino acids, hydrophobic amino acids and aromatic amino acids.

In one aspect, the MIC-1 analogues may comprise substitutions of one or more unnatural and/or non-amino acids, e.g., amino acid mimetics, into the sequence of MIC-1.

Deletions and Truncations.

In one aspect, the MIC-1 analogues of the invention may have one or more amino acid residues deleted from the amino acid sequence of human MIC-1, alone or in combination with one or more insertions or substitutions.

Insertions.

In one aspect, the MIC-1 analogues of the invention may have one or more amino acid residues inserted into the amino acid sequence of human MIC-1, alone or in combination with one or more deletions and/or substitutions.

In one aspect, the MIC-1 analogues of the invention may include insertions of one or more unnatural amino acids and/or non-amino acids into the sequence of MIC-1.

MIC-1 analogues may be described by reference to i) the number of the amino acid residue in the mature MIC-1 protein which corresponds to the amino acid residue which is changed (i.e., the corresponding position in native MIC-1), and to ii) the actual change. In other words, a MIC-1 analogue is a MIC-1 protein in which a number of amino acid residues have been changed when compared to native MIC-1 (SEQ ID NO: 1). These changes may represent, independently, one or more amino acid substitutions, additions, and/or deletions.

The following are non-limiting examples of suitable analogue nomenclature. His6Asp MIC-1 designates an analogue of human native MIC-1, wherein the naturally occurring histidine in position 6 has been substituted with aspartic acid.

As is apparent from the above examples, amino acid residues may be identified by their full name, their one-letter code, and/or their three-letter code. These three ways are fully equivalent.

The term “protein”, as e.g. used in the context of MIC-1 proteins, refers to a compound which comprises a series of amino acids interconnected by amide (or peptide) bonds.

Amino acids are molecules containing an amine group and a carboxylic acid group, and, optionally, one or more additional groups, often referred to as a side chain.

The term “amino acid” includes coded (or proteinogenic or natural) amino acids (amongst those the 20 standard amino acids), as well as non-coded (or non-proteinogenic or non-natural) amino acids. Coded amino acids are those which are naturally incorporated into proteins. The standard amino acids are those encoded by the genetic code. Non-coded amino acids are either not found in proteins, or not produced by standard cellular machinery (e.g., they may have been subject to post-translational modification). In what follows, all amino acids of the MIC-1 proteins for which the optical isomer is not stated is to be understood to mean the L-isomer (unless otherwise specified).

### Alpha-1-Antitrypsin

The present invention relates to the use of alpha-1-antitrypsin (A1AT) as fusion partner for MIC-1. A1AT is a proteinase inhibitor with a molecular size of 394 amino acids and which has a highly ordered globular structure. A1AT exhibits different glycoforms, due to three N-linked glycosylation sites and this confers a naturally occurring heterogeneity to the protein. Several polymorphic variants of A1AT has been detected of which some does not affect biological activity whereas others impairs the function of A1AT leading to human disease. An example of a normal non-pathogenic variant is Val213Ala. An example of a disease causing variant is Glu342Lys. The main function of A1AT is to balance the action of neutrophil-protease enzymes in the lungs, e.g. neutrophil elastase produced by neutrophils in the presence of inflammation. A1AT has been used for replacement therapy for severe lung emphysema. Thus, treatment with A1AT alone is well-proven and toxicology events due to wild type A1AT part of the fusion proteins may be limited. As part of the present invention, A1AT is evaluated as fusion partner for MIC-1 and structural/biophysical variations in the linker regions between A1AT and MIC-1 is explored.

It is well-documented that fusion partners, such as human serum albumin (HSA) or Fc domains (the constant domain of human IgG), can prolong plasma half-life, when fused to the N- or C-terminal of therapeutic proteins. Whereas well-known fusion partners for prolonging plasma half-life, such as HSA and Fc have very long half-life of up to 3 weeks duration, A1AT has a plasma half-life of about 6 days. A1AT does not bind Fc Neonatal receptor to allow recycling from the endosome and will thus most likely confer shorter plasma half-life to MIC-1, when used as fusion partner.

Addition of a large fusion partner to a therapeutic protein may be problematic as the strategy can result in an unstable fusion protein, which is difficult to handle in downstream processing. Thus, the important parameters are expression feasibility, fusion protein stability, maintained biological activity as well as general production cost. Recombinant A1AT proteins comprising a therapeutic protein of interest may be achieved by genetic manipulation, such that the DNA coding for A1AT, or a fragment thereof, is joined to the DNA encoding for the therapeutic protein. A suitable expression host is then transformed or transfected with the fused nucleotide sequences encoded on a suitable plasmid as to express the fusion protein. Human A1AT as fusion partner is thought to increase the plasma half-life of therapeutic proteins through significant size increase which inhibits renal clearance and allows the molecule to be present longer in circulation. Functional variants of A1AT can be designed, which have the same plasma half-life prolonging benefits as the wild-type A1AT (truncated and/or amino acid substituted functional variants). Design of new variants of A1AT may be advantageous, since they can alter the properties of the fusion partner in beneficial ways. Native A1AT comprises a number of glycoforms. In terms of generating homogenous fusion protein products recombinantly in expression hosts it may be of advantage to remove the number of glycosylation sites in the molecule.

A1AT also contains a free Cys at position 232, which may cause aggregation problems and influence biophysical stability of the molecule and which can be removed by amino acid substitution.

A1AT is a biological active proteinase inhibitor with a main function to balance the action of neutrophil-protease enzymes in the lungs, eg neutrophil elastase produced by neutrophils in the presence of inflammation. Whereas the biological activity may be a benefit for the fusion protein, it may also become desirable to remove this activity, if the biological effect of A1AT interferes with the desired pharmacological properties of the MIC-1 fusion protein. This can be accomplished by mutagenesis of the reactive site loop in A1AT in ways, which preserves the advantages connected to production feasibility, stability and prolongation effect, but prevents potential A1AT polymer formation. It may also be desirable to substitute amino acid residues, which may cause challenges for recombinant production, non-limiting examples being Methionine residues and the single Cys232, which are amenable to oxidation

The sequence of the wild-type mature human A1AT is included herein as SEQ ID NO:2 and the sequence is annotated in the Uniprot database with the accession no: P01009. The present invention provides a human A1AT fusion protein comprising, or alternatively consisting of, a biologically active MIC-1 protein or a variant thereof and a biologically active and/or therapeutically active fragment or variant of human A1AT. In one aspect, the invention provides an A1AT fusion protein comprising, or alternatively consisting of, mature native MIC-1 and the mature native human A1AT.

By “functional variant” as used herein is meant a chemical variant of a certain protein which retains substantially the same function as the original protein.

### Fusion Proteins

“Fusion protein” as used herein is intended to mean a hybrid protein expressed by a nucleic acid molecule comprising nucleotide sequences of at least two genes. In one aspect, the fusion proteins of the invention comprise human A1AT as fusion partner fused with native MIC-1 having an activity of pharmaceutical interest. Fusion proteins are often used for improving recombinant expression or stability of therapeutic proteins as well as for improved recovery and purification of such proteins from cell cultures and the like. Fusion proteins may comprise artificial sequences, e.g. a linker sequence.

“Fusion partner” as used herein is intended to mean a protein which is part of a fusion protein, i.e. one of the at least two proteins encompassed by the fusion protein.

In one embodiment of the invention the fusion partner comprises A1AT with an approximate molecular weight of 52 kDa (SEQ ID NO:2) or functional variants thereof, which is operatively linked to the N-terminal of MIC-1 (SEQ ID NO:1) or functional variants thereof with a molecular weight of approximately 12 kDa via an interdomain linker region consisting of amino acid sequences of different length, charges and/or structural motifs.

“Fusion tag” as used herein is intended to mean a protein sequence which is part of a fusion protein, i.e. one of the at least two proteins encompassed by the fusion protein and comprises a sequence which improves expression, solubilisation or purification of the fusion protein, e.g. a 6× Histidine tag (such as His6) or a solubilization domain (such as Thiol:disulfide interchange protein DsbC (DsbC), Maltose Binding Protein (MBP), or Thioredoxin (Trx)).

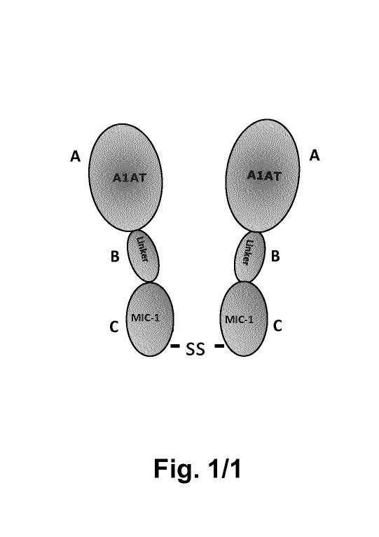

In one aspect of the invention, monomers of NH2-A1AT-linker-MIC-1-COOH with a size of approximately 60 kDa, homodimerizes as the native molecule via interchain disulphide bridge between the two MIC-1 molecules to form an active A1AT-MIC-1 fusion protein with a molecular weight of approximately 120 kDa (depicted as schematic drawing in FIG. 1).

### Peptide Linker

The term “peptide linker” as used herein is intended to mean an amino acid sequence which is typically used to facilitate the function, folding or expression of fusion proteins. It is known in the art that two proteins present in the form of a fusion protein may interact with the functional activities of each other, an interaction that can often be eliminated or reduced by the insertion of a linker between the two protein sequences.

Different exposure of the MIC-1 protein comprised in a fusion protein to its putative receptor, plasma half-life or overall fusion protein stability may be affected by differences in the linker sequence/structure of the fusion protein.

The linkers from the present invention were designed with different predicted biophysical or structural properties comprising variations in the length of the linker (variation of the number of amino acids in the linker), and predicted secondary structure such as alpha-helical structure, rigid structure or flexible, random coil structures or charge or combinations of the above e.g. flexible N-terminal part of linker or rigid N-terminal part of linker combined with alpha-helical or rigid linkers. The present invention includes combinations of linkers containing 1-4 adjacent Pro residues positioned either in the midsection of the linker or in the N-terminal of the linker, which due to conformational rigidity of proline affects the secondary structure in the regions adjacent to the Pro residues. The structure of A1AT indicates that the C-terminal is in close proximity to the core backbone structure of the protein. The linker length may influence the potential interaction between the A1AT and MIC-1 domain by changing the possibility of steric hindrance provided by the fusion partner attached to the biological active MIC-1 domain. The steric hindrance may influence correct folding of the two domains of the fusion protein monomer, formation of the dimer or the interaction of the MIC-1 part with a putative receptor.

### Functional Properties

### Biological Activity—In Vivo Pharmacology

In one aspect the compounds of the invention are potent in vivo, which may be determined as is known in the art in any suitable animal model, as well as in clinical trials.

The non-obese Sprague Dawley rat is one example of a suitable animal model, and the changes in food intake may be determined in such rats in vivo, e.g. as described in Example 2.

In one aspect the compounds of the invention inhibits in vivo food intake in non-obese Sprague Dawley rats.

As an example, in a particular aspect of the invention, the maximum efficacy which is the greatest significant (p<0.10) reduction in 24 hour food intake recorded over 7 days should be at least 10%, or at least 15%.

As an example, in a particular aspect of the invention, the accumulated efficacy which is the sum of significant (p<0.10) reductions in 24 hour food intake compared with vehicle should be at least 10%, or at least 30%.

### Biophysical Properties

In one aspect, the compounds of the invention have good biophysical properties. These properties include but are not limited to physical stability and/or solubility. These and other biophysical properties may be measured using standard methods known in the art of protein chemistry. In a particular embodiment, these properties are improved as compared to native MIC-1 (SEQ ID NO:1). Increased biophysical stability of a fusion protein compared to native MIC-1 may be at least partly be owing to stabilizing effects of the fusion partner.

### Production Processes

Fusion proteins such as those of the present invention may be produced by means of recombinant protein technology known to persons skilled in the art. In general, nucleic acid sequences encoding the proteins of interest or functional variants thereof are modified to encode the desired fusion protein. This modification includes the in-frame fusion of the nucleic acid sequences encoding the two or more proteins to be expressed as a fusion protein. Such a fusion protein can be with or without a linker peptide as well as the fusion protein fused to a fusion tag, e.g. a Histidine tag (such as His6) or a solubilization domain (such as DsbC, MBP or Trx). This modified sequence is then inserted into an expression vector, which is in turn transformed or transfected into the expression host cells.

The nucleic acid construct encoding the fusion protein may suitably be of genomic, cDNA or synthetic origin. Amino acid sequence alterations are accomplished by modification of the genetic code by well-known techniques.

The DNA sequence encoding the fusion protein is usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The vector is preferably an expression vector in which the DNA sequence encoding the fusion protein is operably linked to additional segments required for transcription of the DNA. The term, “operably linked” indicates that the segments are arranged so that they function in concert for their intended purposes, e.g. transcription initiates in a promoter and proceeds through the DNA sequence coding for the polypeptide until it terminates within a terminator.

Thus, expression vectors for use in expressing the fusion protein will comprise a promoter capable of initiating and directing the transcription of a cloned gene or cDNA. The promoter may be any DNA sequence, which shows transcriptional activity in the host cell of choice and may be derived from genes encoding proteins either homologous or heterologous to the host cell.

Additionally, expression vectors for expression of the fusion protein will also comprise a terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

Expression of the fusion protein can be aimed for either intracellular expression in the cytosol of the host cell or be directed into the secretory pathway for extracellular expression into the growth medium.

Intracellular expression is the default pathway and requires an expression vector with a DNA sequence comprising a promoter followed by the DNA sequence encoding the fusion protein followed by a terminator.

To direct the fusion protein into the secretory pathway of the host cells, a secretory signal sequence (also known as signal peptide or a pre sequence) is needed as an N-terminal extension of the fusion protein. A DNA sequence encoding the signal peptide is joined to the 5′ end of the DNA sequence encoding the fusion protein in the correct reading frame. The signal peptide may be that normally associated with the protein or may be from a gene encoding another secreted protein.

The procedures used to ligate the DNA sequences coding for the fusion protein, the promoter, the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

The host cell into which the DNA sequence encoding the fusion protein is introduced may be any cell that is capable of expressing the fusion protein either intracellularly or extracellularly. The fusion protein may be produced by culturing a host cell containing a DNA sequence encoding the fusion protein and capable of expressing the fusion protein in a suitable nutrient medium under conditions permitting the expression of the fusion protein. Non-limiting examples of host cells suitable for expression of fusion proteins are: Escherichia coli, Saccharomyces cerevisiae, as well as human embryonic kidney (HEK), Baby Hamster Kidney (BHK) or Chinese hamster ovary (CHO) cell lines. If posttranslational modifications are needed, suitable host cells include yeast, fungi, insects and higher eukaryotic cells such as mammalian cells.

Once the fusion protein has been expressed in a host organism it may be recovered and purified to the required purity by conventional techniques. Non-limiting examples of such conventional recovery and purification techniques are centrifugation, solubilization, filtration, precipitation, ion-exchange chromatography, immobilized metal affinity chromatography (IMAC), Reversed phase-High Performance Liquid Chromatography (RP-HPLC), gel-filtration and freeze drying.

Examples of recombinant expression and purification of fusion proteins may be found in e.g. Cordingley et al., J. Virol. 1989, 63, pp 5037-5045, Birch et al., Protein Expr Purif., 1995, 6, pp 609-618 and in WO2008/043847.

Examples of microbial expression and purification of fusion proteins may be found in e.g. Chich et al, Anal. Biochem, 1995, 224, pp 245-249 and Xin et al., Protein Expr. Purif. 2002, 24, pp 530-538.

Specific examples of methods of preparing a number of the compounds of the invention are included in the experimental part.

### Mode of Administration

The term “treatment” is meant to include both the prevention and minimization of the referenced disease, disorder, or condition (i.e., “treatment” refers to both prophylactic and therapeutic administration of a compound of the invention or composition comprising a compound of the invention unless otherwise indicated or clearly contradicted by context.

The route of administration may be any route which effectively transports a compound of this invention to the desired or appropriate place in the body, such as parenterally, for example, subcutaneously, intramuscularly or intraveneously. Alternatively, a compound of this invention can be administered orally, pulmonary, rectally, transdermally, buccally, sublingually, or nasally.

The amount of a compound of this invention to be administered, the determination of how frequently to administer a compound of this invention, and the election of which compound or compounds of this invention to administer, optionally together with another pharmaceutically active agent, is decided in consultation with a practitioner who is familiar with the treatment of obesity and related disorders.

### Pharmaceutical Compositions

Pharmaceutical compositions comprising a compound of the invention or a pharmaceutically acceptable salt, amide, or ester thereof, and a pharmaceutically acceptable excipient may be prepared as is known in the art.

The term “excipient” broadly refers to any component other than the active therapeutic ingredient(s). The excipient may be an inert substance, an inactive substance, and/or a not medicinally active substance.

The excipient may serve various purposes, e.g. as a carrier, vehicle, diluent, tablet aid, and/or to improve administration, and/or absorption of the active substance.

The formulation of pharmaceutically active ingredients with various excipients is known in the art, see e.g. Remington: The Science and Practice of Pharmacy (e.g. 19^{th} edition (1995), and any later editions).

The term “physical stability” refers to the tendency of the polypeptide to form biologically inactive and/or insoluble aggregates as a result of exposure to thermo-mechanical stress, and/or interaction with destabilising interfaces and surfaces (such as hydrophobic surfaces). The physical stability of an aqueous polypeptide formulation may be evaluated by means of visual inspection, and/or by turbidity measurements after exposure to mechanical/physical stress (e.g. agitation) at different temperatures for various time periods. Alternatively, the physical stability may be evaluated using a spectroscopic agent or probe of the conformational status of the polypeptide such as e.g. Thioflavin T or “hydrophobic patch” probes.

The term “chemical stability” refers to chemical (in particular covalent) changes in the polypeptide structure leading to formation of chemical degradation products potentially having a reduced biological potency, and/or increased immunogenic effect as compared to the intact polypeptide. The chemical stability can be evaluated by measuring the amount of chemical degradation products at various time-points after exposure to different environmental conditions, e.g. by SEC-HPLC, and/or RP-HPLC.

### Combination Treatment

The treatment with a compound according to the present invention may also be combined with one or more pharmacologically active substances, e.g., selected from antiobesity agents, appetite regulating agents, and agents for the treatment and/or prevention of complications and disorders resulting from or associated with obesity.

### Pharmaceutical Indications

In one aspect, the present invention relates to a compound of the invention, for use as a medicament.

In particular embodiments, the compound of the invention may be used for the following medical treatments:

(i) Prevention and/or treatment of eating disorders, such as obesity, e.g. by decreasing food intake, reducing body weight, suppressing appetite and inducing satiety.

(ii) Prevention and/or treatment of hyperglycemia and/or impaired glucose tolerance.

### Particular Embodiments

The invention is further described by the following non-limiting embodiments of the invention:

1. A compound comprising a fusion protein of formula (I):

A-B-C (I),

wherein
A is human A1AT or a functional variant thereof;
B is a peptide linker, wherein the peptide linker is 10 to 100 amino acids in length; and
C is a MIC-1 protein or an analogue thereof, and
wherein the C-terminus of A1AT or a functional variant thereof is fused to the N-terminus of the peptide linker, and the C-terminus of the peptide linker is fused to the N-terminus of the MIC-1 protein or analogue thereof.
2. A compound according to embodiment 1, wherein the peptide linker is 25-100, 25-50 or 30-50 amino acids in length.
3. A compound according to embodiments 1-2, wherein the peptide linker comprises the amino acid sequence [X-Yₘ]ₙ, wherein X is Asp or Glu; Y is Ala; m is from 2 to 4; and n is at least 5.
4. A compound according to any one of the preceding embodiments, wherein the compound is a homodimer of two fusion proteins of formula (I):

A-B-C (I)

formed by an interchain disulphide bridge between the two MIC-1 proteins or analogues thereof.
5. A compound according to any one of the preceding embodiments, wherein X is Asp.
6. A compound according to any one of the preceding embodiments, wherein X is Glu.
7. A compound according to embodiments 3-6, wherein m is 2-3 and n is 5-12.
8. A compound according to embodiments 3-7, wherein n is 10.
9. A compound according to embodiments 3-7, wherein n is 12.
10. A compound according to any of the preceding embodiments comprising Pro residues in the linker.
11. A compound according to any of the preceding embodiments comprising 1-4 Pro residues in the linker.
12. A compound according to embodiment 11, comprising 4 Pro residues in the linker.
13. A compound according to embodiment 11, comprising 3 Pro residues in the linker.
14. A compound according to embodiment 11, comprising 2 Pro residues in the linker.
15. A compound according to embodiment 11, comprising 1 Pro residue in the linker.
16. A compound according to embodiments 10-15, wherein the Pro residues are positioned in the N-terminal of the linker.
17. A compound according to embodiments 10-15, wherein the Pro residues are not positioned in the terminals of the linker.
18. A compound according to embodiments 10-15, comprising 1-4 Pro residues in a midsection of the linker covering from approximately 20%-30% of the linker length.
19. A compound according to embodiment 18 comprising 2 Pro residues in a midsection of the linker covering from approximately 20%-30% of the linker length.
20. A compound according to embodiment 18 comprising 1 Pro residues in a midsection of the linker covering from approximately 20%-30% of the linker length.
21. A compound according to embodiments 10-15, comprising 1-4 Pro residues in a midsection of the linker covering from approximately 10%-20% of the linker length.
22. A compound according to embodiment 21 comprising 2 Pro residues in a midsection of the linker covering from approximately 10%-20% of the linker length.
23. A compound according to embodiment 21 comprising 1 Pro residue in a midsection of the linker covering from approximately 10%-20% of the linker length.
24. A compound according to embodiments 10-15, comprising 1-4 Pro residues in a midsection of the linker covering from approximately 5%-10% of the linker length.
25. A compound according to embodiment 24 comprising 2 Pro residues in a midsection of the linker covering from approximately 5%-10% of the linker length.
26. A compound according to embodiment 24 comprising 1 Pro residues in a midsection of the linker covering from approximately 5%-10% of the linker length.
27. A compound according embodiments 1-9, wherein the peptide linker comprises EAAEAAEAAEAAEAAEAAEAAEAAEAAEAAEE (SEQ ID NO: 8).
28. A compound according to embodiments 1-9, wherein the peptide linker comprises GGSSEAAEAAEAAEAAEAAEAAEAAEAAEAAEAAEE (SEQ ID NO: 9).
29. A compound according to embodiments 1-9, wherein the peptide linker comprises PPPPEAAEAAEAAEAAEAAEAAEAAEAAEAAEAAEE (SEQ ID NO:10).
30. A compound according to embodiments 1-9, wherein the peptide linker comprises PPPEAAEAAEAAEAAEAAEAAEAAEAAEAAEAAEE (SEQ ID NO: 13).
31. A compound according to embodiments 1-9, wherein the peptide linker comprises PPEAAEAAEAAEAAEAAEAAEAAEAAEAAEAAEE (SEQ ID NO: 14).
32. A compound according to embodiments 1-9, wherein the peptide linker comprises EAAEAAEAAEAAEAAEAAEPEAAEAAEAAEAAEAAEAAE (SEQ ID NO: 15).
33. A compound according to embodiments 1-9, wherein the peptide linker comprises EAAEAAEAAEAAEAAEAAEPPEAAEAAEAAEAAEAAEAAE (SEQ ID NO: 16).
34. A compound according to embodiments 1-9, wherein the peptide linker comprises EAAAEAAAEAAAEAAAEAAAEAAAEAAAEE (SEQ ID NO: 17).
35. A compound according to any one of the preceding embodiments, wherein C is an analogue of MIC-1 displaying at least 85% sequence identity to native MIC-1 (SEQ ID NO:1).
36. A compound according to any one of the preceding embodiments, wherein C is an analogue of MIC-1 displaying at least 90% sequence identity to native MIC-1 (SEQ ID NO:1).
37. A compound according to any one of the preceding embodiments, wherein C is an analogue of MIC-1 displaying at least 95% sequence identity to native MIC-1 (SEQ ID NO:1).
38. A compound according to any one of the preceding embodiments, wherein C is an analogue of MIC-1 having a maximum of 17 amino acid modifications compared to native MIC-1 (SEQ ID NO:1).
39. A compound according to any one of the preceding embodiments, wherein C is an analogue of MIC-1 having a maximum of 11 amino acid modifications compared to native MIC-1 (SEQ ID NO:1).
40. A compound according to any one of the preceding embodiments, wherein C is an analogue of MIC-1 having a maximum of 5 amino acid modifications compared to native MIC-1 (SEQ ID NO:1).
41. A compound according to any one of the preceding embodiments, wherein C is an analogue of MIC-1 in which Asn3 is substituted with Glu (SEQ ID NO:19).
42. A compound according to embodiments 1-40, wherein C is mature human MIC-1 (SEQ ID NO:1).
43. A compound according to any one of the preceding embodiments, wherein A is a functional variant of human A1AT displaying at least 85% sequence identity to wild type human A1AT (SEQ ID NO:2).
44. A compound according to any one of the preceding embodiments, wherein A is a functional variant of human A1AT displaying at least 90% sequence identity to wild type human A1AT (SEQ ID NO:2).
45. A compound according to any one of the preceding embodiments, wherein A is a functional variant of human A1AT displaying at least 95% sequence identity to wild type human A1AT (SEQ ID NO:2).
46. A compound according to any one of the preceding embodiments, wherein A is a functional variant of human A1AT comprising another amino acid than cysteine in the position corresponding to position 232 of wild type human A1AT (SEQ ID NO:2).
47. A compound according to embodiments 1-45, wherein A is a functional variant of human A1AT comprising alanine or serine in the position corresponding to position 232 of wild type human A1AT (SEQ ID NO:2).
48. A compound according to embodiments 1-45, wherein A is a functional variant of human A1AT comprising alanine in the position corresponding to position 232 of wild type human A1AT (SEQ ID NO:2).
49. A compound according to embodiments 1-45, wherein A is wild type human A1AT of SEQ ID NO:2.
50. A compound according to any one of the preceding embodiments, further comprising a fusion partner.
51. A compound according to any one of the preceding embodiments, further comprising an N-terminal fusion partner.
52. A compound according to any one of the preceding embodiments, wherein said compound is a MIC-1 agonist.
53. A compound according to any one of the preceding embodiments, wherein said compound is capable of decreasing food intake.
54. A compound according to any one of the preceding embodiments, wherein said compound has the effect in vivo of decreasing food intake determined in a single-dose study in non-obese Sprague Dawley rats.
55. A compound according to claim 1, wherein A is human A1AT of SEQ ID NO:2, B is the peptide linker of SEQ ID NO:15, and C is native human MIC-1 of SEQ ID NO:1.
56. A compound according to claim 1, wherein A is human A1AT of SEQ ID NO:2, B is the peptide linker of SEQ ID NO:16, and C is native human MIC-1 of SEQ ID NO:1.
57. A compound according to claim 1, wherein A is human A1AT of SEQ ID NO:20, B is the peptide linker of SEQ ID NO:15, and C is native human MIC-1 of SEQ ID NO:19.
58. A compound according to claim 1, wherein A is human A1AT of SEQ ID NO:2, B is the peptide linker of SEQ ID NO:17, and C is native human MIC-1 of SEQ ID NO:1.
59. A compound according to claim 1, consisting of a His-tagged fusion protein of formula (I), wherein the His-tag is the His-tag of SEQ ID NO:3, A is human A1AT of SEQ ID NO:2, B is the peptide linker of SEQ ID NO:15, and C is native human MIC-1 of SEQ ID NO:1.
60. A compound according to claim 1, consisting of a His-tagged fusion protein of formula (I), wherein the His-tag is the His-tag of SEQ ID NO:3, A is human A1AT of SEQ ID NO:2, B is the peptide linker of SEQ ID NO:16, and C is native human MIC-1 of SEQ ID NO:1.
61. A compound according to claim 1, consisting of a His-tagged fusion protein of formula (I), wherein the His-tag is the His-tag of SEQ ID NO:3, A is human A1AT of SEQ ID NO:20, B is the peptide linker of SEQ ID NO:15, and C is native human MIC-1 of SEQ ID NO:19.
62. A compound according to claim 1, consisting of a His-tagged fusion protein of formula (I), wherein the His-tag is the His-tag of SEQ ID NO:3, A is human A1AT of SEQ ID NO:2, B is the peptide linker of SEQ ID NO:17, and C is native human MIC-1 of SEQ ID NO:1.
63. A pharmaceutical composition comprising a compound according to any one of embodiments 1-62 or a pharmaceutically acceptable salt, amide or ester thereof, and one or more pharmaceutically acceptable excipients.
64. A compound according to any one of embodiments 1-62 for use as a medicament.
65. A compound according to any one of embodiments 1-62 for use in the prevention and/or treatment of eating disorders, such as obesity, e.g. by decreasing food intake, reducing body weight, suppressing appetite and inducing satiety.
66. A compound according to any one of embodiments 1-62 for use in the prevention and/or treatment of obesity.
67. The use of a compound according to any one of embodiments 1-62 in the manufacture of a medicament for the treatment of eating disorders, such as obesity, e.g. by decreasing food intake, reducing body weight, suppressing appetite and inducing satiety.
68. The use of a compound according to any one of embodiments 1-62 in the manufacture of a medicament for the treatment of obesity.
69. A method of treating or preventing eating disorders, such as obesity, e.g. by decreasing food intake, reducing body weight, suppressing appetite and inducing satiety by administering a pharmaceutically active amount of a compound according to any one of embodiments 1-62.
70. A method of treating or preventing obesity by administering a pharmaceutically active amount of a compound according to any one of embodiments 1-62.
71. A polynucleotide molecule encoding a compound according to any one of embodiments 1-62.

### EXAMPLES

This experimental part starts with a list of abbreviations, and is followed by a section including general methods of preparation, purification and characterisation of the compounds of the invention. Then follows an example relating to the activity and properties of these fusion proteins (section headed pharmacological methods). The examples serve to illustrate the invention.

### LIST OF ABBREVIATIONS

“Main peak” refers to the peak in a purification chromatogram which has the highest UV intensity in milliabsorbance units and which contains the fusion protein.
HPLC is High performance liquid chromatography.
SDS-PAGE is Sodium dodecyl sulfate Polyacrylamide gel electrophoresis.
IMAC is immobilized metal affinity chromatography.
SEC is size exclusion chromatography.
MS is mass spectrometry.

### Materials and Methods

In short, the general design of the fusion proteins comprised of 3 parts; human A1AT in the N-terminus, a linker region and wild type MIC-1 at the C-terminus. The plasmid backbone pTT5 suitable for expression in mammalian cells was used in the present invention and has been described previously (Shi et al, Biochemistry 2005, 44, 15705-15714). In addition to the fusion protein coding sequence, all plasmids encoded a CD33 secretion signal sequence directly upstream of the region encoding the N-terminal part of the fusion protein allowing the recombinant fusion protein to be secreted into the growth medium. To facilitate protein purification, all constructs contained a 6×His-tag (His6) N-terminally on the fusion protein. A wide range of different amino acid linkers were tested with the above fusion partners to see whether they affect overall yields of the proteins.

The fusion proteins were prepared by state of the art recombinant protein technology methods. Plasmids encoding fusion proteins were obtained using gene synthesis based strategies of the relevant sequences and subcloning into pTT5 mammalian expression vectors (Genscript Inc) and expression yields were initially evaluated in small scale volumes using Human Kidney Embryonic cells (HEK) (Expi293F, Invitrogen) as expression host system. The expressed fusion proteins were scaled up in shaker flasks and purified using two step automated chromatography comprised of capture on a HisTrap Excel column followed by size exclusion chromatography (SEC) on a Superdex 200 column. Fractions were pooled to avoid contamination from product-related impurities eluting before the main peak.

The methods are described in more detail below.

### General Methods of Preparation

### Small Scale Screening and Expression of Fusion Constructs

Expression levels for each construct were determined by transient transfection of the plasmids into Human Embryonic Kidney (HEK) cells (Expi293F™, Life Technologies™ #A14527) in 2 ml suspension cultures grown in Expi293™ Expression Medium (Life Technologies™ #A1435101). The expi293 cells were grown in disposable 24-well multiwell blocks (Axygen, #P-DW-10 ml-24-C-S) at 37° C., 8% CO₂ and 80% humidity. The shaking speed was 200 rpm in an Infors Multitron Cell incubator with a 50 mm orbital throw. For each transfection, 2 μg DNA in 100 ul of transfection medium (Opti-MEM® I (1×)+GlutaMAX™-I Reduced Serum Medium, Life Technologies™ #51985-026) and 5.4 μl ExpiFectamine™ 293 reagent (ExpiFectamine™ 293 Transfection Kit, Life Technologies™ #A14525) in transfection medium were used, according to the manufacturer's instructions. 16-20 hours after transfection, the cultures were fed with 10 μl enhancer 1 and 100 μl enhancer 2 (ExpiFectamine™ 293 Transfection Kit, Life Technologies™ #A14525). Four to six days after transfection, the cell cultures were harvested by centrifugation at 4000 g for 10 minutes, and the clarified culture medium used for further analysis of protein expression.

The production feasibility of each fusion protein was assessed by a small scale purification screen using an immobilized metal affinity chromatography (IMAC) step. The purified protein solutions were visualized by running samples on SDS-PAGE (Sodium dodecyl sulfate Polyacrylamide gel electrophoresis) gels (Novex® NuPAGE® 4-12% Bis-Tris midi protein gels, 26 wells, Life Technologies™ #WG1403BOX) without sample reduction, and the resulting protein bands visualized by Coomassie staining (InstantBlue™, Expedeon #ISBL1L). The results were used to determine the cell culture volume needed of each construct to provide enough protein for in vivo assessment of efficacy.

### Scale-Up Expression of A1AT-MIC-1 Fusion Proteins.

The plasmids encoding MIC-1 fusion proteins were transformed to OneShot® Top10F′ chemically competent E. coli cells (OneShot® Top10F′, Life Technologies™ #C303003, alternatively XL1-Blue Competent Cells, Agilent technologies, #200249), colonies were grown on Amp/Carb selective agar plates and transformants used to inoculate liquid Terrific Broth (TB) cultures. After overnight growth, the pelleted E. coli cells were used for large scale plasmid preparations (EndoFree® Plasmid Mega Kit, Qiagen® #12381).

Transient expression was performed by adding plasmid DNA (1 mg/litre cell culture) in OptiMEM® transfection medium (50 ml/litre cell culture) to ExpiFectamine™ 293 reagent (2.7 ml/litre cell culture) in OptiMEM® transfection medium (50 ml/litre cell culture), incubating for 10 to 20 minutes and then adding the transfection mix to the cell culture (expi293F cells at 3×106 cells/imp. 16 to 20 hours after transfection, the cultures were fed with enhancers 1 (5 ml/litre cell culture) and enhancer 2 (50 ml/litre cell culture). The expi293 cells were grown in 1 litre disposable shaker flasks (Corning #CLS431147) at 37° C., 8% CO₂ and 80% humidity. The shaking speed was 110 rpm in an Infors Multitron Cell incubator with a 50 mm orbital throw.

Approximately 90-120 hours after transfection the cultures were harvested by centrifugation at 4000 g for 10 minutes. The clarified medium was sterile filtered through a 0.22 uM filter before purification.

### Purification

Following centrifugation and filtration through a 0.22 μm filter the clarified supernatant was conditioned for IMAC purification by addition of 200 mL His-binding buffer (300 mM Sodium Phosphate (NaP), 1.8 M NaCl, 60 mM imidazole, pH 7.5) per liter supernatant.

Using an ÄKTAxpress chromatography system, the conditioned supernatant was applied at low flowrate of 1 ml/minute to a 5 ml HisTrap Excel column (GE-Healthcare, Sweden) equilibrated in Buffer A (50 mM NaP, 300 mM NaCl, 10 mM Imidazole, pH 7.5) after which low affinity binding impurities were eluted with Wash Buffer (50 mM NaP, 300 mM NaCl, 30 mM Imidazole, pH 7.5). Bound fusion protein was step eluted with 100% Buffer B (50 mM NaP, 300 mM NaCl, 300-500 mM Imidazole, pH 7.5) and the main peak was collected using the peak detection option of the Unicorn™ software and automatically purified further using preparative size exclusion chromatography (SEC) in 1×PBS pH 7.4 (Ampliqon) on a HiLoad Superdex 200 16/600 PG column (GE-Healthcare, Sweden). 1.8 ml fractions were collected and analyzed by non-reducing SDS-PAGE using precast 4-12% NuPAGE® gels (Life Technologies™). In short the samples were mixed with 4×LDS (lithium dodecyl sulphate) sample buffer. The mixture was heated 5 minutes at 95° C. before loading the SDS-PAGE gels. Novex SeeBlue® plus2 pre-stained Protein standard (Life Technologies™) were run alongside the fractions on SDS-PAGE for size estimation. Protein was visualized using InstantBlue™ stain (Expedeon, Cambridgeshire, UK) according to the manufacturer instructions.

### General Methods of Detection and Characterisation

### MS Analysis

Peptide mass mapping was performed to verify correct linker sequences and was done using methods know to persons skilled in the art. In short, purified proteins were subjected to enzymatic digestion using a method adopted from “In solution tryptic digest and guanidation kit”, Pierce product nr. 89895. The enzymes used were trypsin or AspN. Peptide masses were obtained with Thermo-Dionex Ultimate3000™ HPLC (Thermo Fisher Scientific) coupled to a Maxis Impact™ ESI-Q-OTOF mass spectrometer (Bruker Daltonics) and using a Reversed phased column (Waters BEH300 C8 1.7 μm 1.0×100) at a temperature of 45° C. and a flow of 0.2 ml/min and a 0-90% acetonitrile/TFA gradient with solvents composed of Buffer A: 0.1 TFA/99.9% water and Buffer B: 0.1% TFA/99.9 Acetonitrile designed to for separation of the peptides. Peptide mass mapping to allow identification and verification of the correct linker linker sequence in the fusion proteins was done using the Data analysis software (Bruker Daltonics) to extract experimental determined masses of peptides and the Biotools software (Biotools) for matching experimental masses against the calculated masses derived from the expected fusion protein sequences according to the manufacturer's instructions. In general, variable modifications were set to “Oxidation (M)” and “Carbamidomethyl (C) and the mass tolerance was set to 20 ppm and MS/MS tolerance to 50 mmu.

Chemiluminescent Nitrogen Detection (CLND) coupled to a standard HPLC was used to determine the protein concentration.

### Example 1: Expression and Purification of the Compounds of the Invention

The different plasmids encoding the fusion protein variants depicted in Table 1 were designed with differences in the linker sequence between A1AT and MIC-1. Plasmids were generated by well-known recombinant DNA technology methods (obtained from GenScript Inc). The linker sequence is given in Table 2.

As a representative example, large scale production of Compound 5 was performed by transient expression in Expi293F cells as described in materials and methods section. Briefly, 500 μg plasmid DNA was added to 25 ml of Opti-MEM® transfection medium and 1.35 ml ExpiFectamine™ 293 reagent was added to 25 ml of Opti-MEM® transfection medium. The two solutions were combined to form a transfection mix. After 20 minutes incubation, the transfection mix was added to 500 ml of expi293F cell culture with a cell density of 3×106 cells/ml. 18 hours after transfection, the cultures were fed with 2.5 ml of enhancer 1 and 25 ml of enhancer 2. Five days after transfection the culture was harvested by centrifugation at 4000 g for 10 minutes. The clarified medium was sterile filtered through a 0.22 uM filter before purification.

To examine the in vivo effect of fusing a A1AT molecule to the N-terminus of the MIC-1 protein by variable linkers the expressed molecule were purified using the method described in the materials sections. Compound 5 was successfully purified using automated immobilized metal ion chromatography coupled to size exclusion. Two peaks within the total volume of the SEC column were fractioned and analysed. The first peak eluted at the void of the column and non-reducing SDS-PAGE confirmed the aggregated state of the eluted protein. The main peak partially overlapped with the aggregate peak. Therefore, not all fractions representing the entire main peak were included in the pool. Non-reducing SDS-PAGE of the pooled fractions resulted in a single band which migrated as a ˜120 kDa protein.

TABLE 1
List of compounds of the present invention. Compounds referred to in the table
has wild type A1AT (SEQ: ID NO: 2) or Cys232Ala A1AT (SEQ ID NO: 20) in the N-
terminal, an intervening linker sequence as indicated with an amino acid sequence and
the wild type MIC-1 sequence (SEQ ID NO: 1) or a Asn3Glu analog of wild type MIC-1
(SEQ ID NO: 19) in the C-terminal (SEQ ID NO: 1). An N-terminal His6 tag was
included for all constructs to facilitate IMAC purification (SEQ ID NO: 3). Compounds
1-4 were included for comparison.

| | | N- | | | |
|---|---|---|---|---|---|
| | | terminal | | Linker | MIC-1 |
| Compound | | His-tag | A1AT | sequence | Protein |
| 1 | A1AT-SG-MIC1 | SEQ ID | SEQ ID | SEQ ID | SEQ ID |
| | | NO: 3 | NO: 2 | NO: 4 | NO: 1 |
| 2 | A1AT-(GS)32-MIC1 | SEQ ID | SEQ ID | SEQ ID | SEQ ID |
| | | NO: 3 | NO: 2 | NO: 5 | NO: 1 |
| 3 | A1AT-PPPP(GS)28-MIC1 | SEQ ID | SEQ ID | SEQ ID | SEQ ID |
| | | NO: 3 | NO: 2 | NO: 6 | NO: 1 |
| 4 | A1AT-(PT)16P-MIC1 | SEQ ID | SEQ ID | SEQ ID | SEQ ID |
| | | NO: 3 | NO: 2 | NO: 7 | NO: 1 |
| 5 | A1AT-(EAA)10EE-MIC1 | SEQ ID | SEQ ID | SEQ ID | SEQ ID |
| | | NO: 3 | NO: 2 | NO: 8 | NO: 1 |
| 6 | A1AT-GGSS(EAA)10EE-MIC1 | SEQ ID | SEQ ID | SEQ ID | SEQ ID |
| | | NO: 3 | NO: 2 | NO: 9 | NO: 1 |
| 7 | A1AT-PPPP(EAA)10EE-MIC1 | SEQ ID | SEQ ID | SEQ ID | SEQ ID |
| | | NO: 3 | NO: 2 | NO: 10 | NO: 1 |
| 8 | A1AT-AE-MIC1 | SEQ ID | SEQ ID | SEQ ID | SEQ ID |
| | | NO: 3 | NO: 2 | NO: 11 | NO: 1 |
| 9 | A1AT-PT-MIC1 | SEQ ID | SEQ ID | SEQ ID | SEQ ID |
| | | NO: 3 | NO: 2 | NO: 12 | NO: 1 |
| 10 | A1AT-PPP(EAA)10EE-MIC1 | SEQ ID | SEQ ID | SEQ ID | SEQ ID |
| | | NO: 3 | NO: 2 | NO: 13 | NO: 1 |
| 11 | A1AT-PP(EAA)10EE-MIC1 | SEQ ID | SEQ ID | SEQ ID | SEQ ID |
| | | NO: 3 | NO: 2 | NO: 14 | NO: 1 |
| 12 | A1AT-(EAA)6P(EAA)6-MIC1 | SEQ ID | SEQ ID | SEQ ID | SEQ ID |
| | | NO: 3 | NO: 2 | NO: 15 | NO: 1 |
| 13 | A1AT-(EAA)6PP(EAA)6-MIC1 | SEQ ID | SEQ ID | SEQ ID | SEQ ID |
| | | NO: 3 | NO: 2 | NO: 16 | NO: 1 |
| 14 | A1AT-(EAA)6P(EAA)6E- | SEQ ID | SEQ ID | SEQ ID | SEQ ID |
| | MIC1_N3E | NO: 3 | NO: 2 | NO: 15 | NO: 19 |
| 15 | A1AT(C-A)-(EAA)6P(EAA)6E- | SEQ ID | SEQ ID | SEQ ID | SEQ ID |
| | MIC1_N3E | NO: 3 | NO: 20 | NO: 15 | NO: 19 |
| 16 | A1AT-(EAAA)7EE-MIC1 | SEQ ID | SEQ ID | SEQ ID | SEQ ID |
| | | NO: 3 | NO: 2 | NO: 17 | NO: 1 |
| 17 | A1AT-(QAA)10QQ-MIC1 | SEQ ID | SEQ ID | SEQ ID | SEQ ID |
| | | NO: 3 | NO: 2 | NO: 18 | NO: 1 |

TABLE 2
List of peptide linkers with corresponding SEQ ID
NO and amino acid sequence.

| SEQ ID NO | Linker sequence |
|---|---|
| SEQ ID NO: 4 | GGSSSGSGGSGGSGSGGSGGSGS |
| SEQ ID NO: 5 | GGSSSGSGGSGGSGSGGSGGSGSGSGGSGGSG |
| SEQ ID NO: 6 | PPPPSGSGGSGGSGSGGSGGSGSGSGGSGGSG |
| SEQ ID NO: 7 | PTPTPTPTPTPTPTPTPTPTPTPTPTPTPTPTP |
| SEQ ID NO: 8 | EAAEAAEAAEAAEAAEAAEAAEAAEAAEAAEE |
| SEQ ID NO: 9 | GGSSEAAEAAEAAEAAEAAEAAEAAEAAEAAEAA |
| | EE |
| SEQ ID NO: 10 | PPPPEAAEAAEAAEAAEAAEAAEAAEAAEAAEAA |
| | EE |
| SEQ ID NO: 11 | GGSSEAAEAAEAAEAAEAAEAAE |
| SEQ ID NO: 12 | GGSSPTPTPTPTPTPTPTPTPTP |
| SEQ ID NO: 13 | PPPEAAEAAEAAEAAEAAEAAEAAEAAEAAEAAEE |
| SEQ ID NO: 14 | PPEAAEAAEAAEAAEAAEAAEAAEAAEAAEAAEE |
| SEQ ID NO: 15 | EAAEAAEAAEAAEAAEAAEPEAAEAAEAAEAAEA |
| | AEAAE |
| SEQ ID NO: 16 | EAAEAAEAAEAAEAAEAAEPPEAAEAAEAAEAAE |
| | AAEAAE |
| SEQ ID NO: 17 | EAAAEAAAEAAAEAAAEAAAEAAAEAAAEE |
| SEQ ID NO: 18 | QAAQAAQAAQAAQAAQAAQAAQAAQAAQAAQQ |

TABLE 3
List of MIC-1 variants with corresponding SEQ ID NO.

| SEQ ID NO | MIC-1 variants |
|---|---|
| SEQ ID NO: 1 | Wild type human MIC-1 (hMIC-1) |
| SEQ ID NO: 19 | Asn3Glu hMIC-1 |

TABLE 4
List of alpha 1 antitrypsin (A1AT) variants with corresponding
SEQ ID NO.

| SEQ ID NO | Alpha 1 antitrypsin variants |
|---|---|
| SEQ ID NO: 2 | Wild type A1AT |
| SEQ ID NO: 20 | Cys232Ala A1AT |

### Pharmacological Methods

### Example 2: Effect of Fusions Proteins of the Invention on Food Intake in Lean Sprague Dawley Rats

The purpose of this example is to test the efficacy of the compounds in vivo.

The in vivo efficacy of the compounds of the invention was measured in 9-11 weeks old non-obese Sprague Dawley rats. Animals were injected once with a dose of 4 nmol/kg body weight. Compounds were administrate subcutaneously (1 ml/kg) in a physiological isotonic phosphate buffered saline (PBS) solution (137 mM NaCL; 2.7 mM KCl; 10 mM Na₂HPO₄; 1.8 mM KH₂PO₄). Changes in food intake were measured by an automatic food monitoring system (HM2 BioDAQ or Feedwin systems Ellegaard A/S for rat,). Prior to housing in the HM2 system rats had a chip inserted in the dorsal neck region for monitoring in the system. Rats in the HM2 system were house three per cage. In the Feedwin system rats were single housed. In the BioDAQ system rats were housed two per cage separated by a divider. Each compound was tested in n=5 animals. Animals were acclimatized for at least for 7 days prior to the experiment. Collected data (see Table 5) are expressed as daily food intake (24 hour food intake) measured from the onset of each daily 12 hour dark phase to the next dark phase. Daily changes in food intake in response to administrated compound were calculated by subtracting the average daily food intake of the vehicle/PBS group from the average daily food intake of the treatment group. Changes were considered significant if p<0.1 using a student's t-test (two-tailed). Results are expressed as the “maximum reduction” in food intake compared with vehicle [percentage] at any given day; Data are also expressed as the “accumulated reduction” in food intake which as the sum of significant reductions (p<0.1) in food intake [percentage] over the study period; Data are also expressed as the “duration of effect” which is the number of days with a significant reduction (p<0.1) in food intake.

Compounds 1-3 did not show any significant PD effects on either maximum efficacy or accumulated food-intake (Table 1, Table 5). Since these three linkers are based on Gly and Ser residues, data indicates that typical flexible linkers of different lengths, even up to above 30 aa, does not result in a functionally active variant of A1AT-MIC-1. In addition, Compound 4 and 9 comprising Pro and Thr residues, did also not show any significant decrease of food intake (Table 1, Table 5). On the contrary, Compound 5 significantly reduced food intake after 24 hours of dosing lasting 2 days with a maximum efficacy of 18% and an accumulated food intake of 35%. Compounds 5, 6 and 7 suppressed food intake 24 hours after a single dose of 4 nmol/kg in lean SD rats with 18%, 19% and 15%, respectively (Table 1, Table 5). Compound 16 comprising 7 Glu-Ala-Ala-Ala repeats also resulted in significant reduction in food intake. Altogether these data show that these four linkers comprising variants of repeats of Glu and Ala residues, results in biological active fusion proteins of A1AT and MIC-1, which significantly reduces food-intake and in case of Compound 16 with an extended duration effect of 3 days. If Glu residues of Compound 5 linker was replaced by Gln residues (Compound 17) or the number of Glu/Ala repeats were reduced from 10 to 6 (Compound 8) no significant effect on food intake was observed. These data altogether shows that A1AT-MIC-1 fusion proteins of the invention must contain both an aa structure based on Glu and Ala repeats and a minimum length of the linker in order to become biological efficacious in reducing food intake (Table 1, Table 5).

Compounds 10, 11 containing Pro residues in the N-terminal of Glu/Ala repeat containing linkers reduced food intake with 21% (maximum efficacy) and Compound 12 and 13 containing 1 or 2 Pro in the middle of the Glu/Ala repeat containing linker, respectively, reduced food intake with 28% and 23% (maximum efficacy). Data shows that variations in the number of adjacent Pro residues or their position in the linker can affect maximum efficacy, accumulated efficacy as well as duration of effect on food intake, if combined with Glu/Ala repeat containing linkers. On the contrary, no significant effect on food intake was observed when combining Pro residues in the N-terminal with flexible Gly/Ser rich linkers (exemplified by compound 3 compared with compound 7).

Results with compound 14 and 15 shows, that potent linkers of the invention can still result in significant effect on food intake when combined with functional variants of MIC-1 and A1AT, as exemplified by substitution of Asn3 in MIC-1 (Compound 14, Table 3) and Cys232 in A1AT (Compound 15, Table 4).

TABLE 5
Effect of a single dose (4 nmol/kg) of A1AT-MIC-1 analogues
on daily food intake in lean SD rats. Data are expressed
in 3 ways, 1) maximum efficacy which is the greatest significant
(p < 0.10) reduction in 24 hours food intake recorded over
the 7 days, 2) Accumulated efficacy which is the sum of significant
(p < 0.10) reductions in 24 hours food intake compared with
vehicle and 3) Duration of effect which is the number of days
with a significant (p < 0.1) reduction in food intake compared
with vehicle. Compounds 1-4 were included for comparison.

| | Maximum | Accumulated | Duration of |
|---|---|---|---|
| Compound | efficacy | efficacy | effect |
| 1 | 0 | 0 | 0 |
| 2 | 0 | 0 | 0 |
| 3 | 0 | 0 | 0 |
| 4 | 0 | 0 | 0 |
| 5 | 18 | 35 | 2 |
| 6 | 19 | 19 | 1 |
| 7 | 15 | 15 | 1 |
| 8 | 0 | 0 | 0 |
| 9 | 0 | 0 | 0 |
| 10 | 21 | 40 | 2 |
| 11 | 21 | 21 | 1 |
| 12 | 28 | 65 | 4 |
| 13 | 23 | 49 | 3 |
| 14 | 18 | 30 | 2 |
| 15 | 23 | 39 | 2 |
| 16 | 18 | 50 | 3 |
| 17 | 0 | 0 | 0 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound comprising a fusion protein of formula (I):
A-B-C (I),
wherein A is human A1AT or a functional variant thereof;
wherein B is a peptide linker that is 10 to 100 amino acids in length;
wherein C is a MIC-1 protein or an analogue thereof;
wherein the C-terminus of the A1AT or a functional variant thereof is fused to the N-terminus of the peptide linker, and
wherein the C-terminus of the peptide linker is fused to the N-terminus of the MIC-1 protein or analogue thereof.

2. The compound according to claim 1, wherein the peptide linker comprises the amino acid sequence [X-Yₘ]ₙ, wherein X is Asp or Glu; Y is Ala; m is from 2 to 4; and n is at least 5.

3. The compound according to claim 2, wherein X is Glu.

4. The compound according to claim 2, wherein m is 2-3 and n is 5-12.

5. The compound according to claim 2, wherein n is 7-12.

6. The compound according to claim 2, wherein the peptide linker comprises 1-4 Pro residues.

7. The compound according to claim 6, wherein the 1-4 Pro residues are in a midsection of the linker covering from approximately 20%-30% of the linker length.

8. The compound according to claim 6, wherein the peptide linker comprises 2 Pro residues.

9. The compound according to claim 8, wherein the peptide linker comprises 1 Pro residue.

10. The compound according to claim 1, wherein C is an analogue of MIC-1 displaying at least 85% sequence identity to native MIC-1 (SEQ ID NO:1).

11. The compound according to claim 1, wherein A is a functional variant of human A1AT displaying at least 85% sequence identity to wild type human A1AT (SEQ ID NO:2).

12. The compound according to claim 1, wherein the peptide linker is 25 to 100 amino acids in length.

13. A pharmaceutical composition comprising a compound according to claim 1 or a pharmaceutically acceptable salt, amide or ester thereof, and one or more pharmaceutically acceptable excipients.

14. (canceled)

15. A method of treating an eating disorder, comprising administering to a subject in need thereof the compound according to claim 1.
